(19) United States
(12) Patent Application Publication
(10) Pub. No.: US 2025/0157630 A1
(43) Pub. Date: May 15, 2025

(54) SYSTEMS, DEVICES, AND METHODS FOR MAGNETIC RESONANCE IMAGING

(71) Applicant: WUHAN UNITED IMAGING LIFE SCIENCE INSTRUMENT CO., LTD., Wuhan (CN)

(72) Inventors: Yao XING, Shanghai (CN); Meiling JI, Wuhan (CN); Renkuan ZHAI, Wuhan (CN); Chuang YANG, Wuhan (CN); Guocan SHANG, Wuhan (CN)

(73) Assignee: WUHAN UNITED IMAGING LIFE SCIENCE INSTRUMENT CO., LTD., Wuhan (CN)

(21) Appl. No.: 18/948,424

(22) Filed: Nov. 14, 2024

(30) Foreign Application Priority Data
Nov. 14, 2023 (CN) 202311538169.8

(51) Int. Cl. G16H 30/20

(52) U.S. Cl. CPC G16H 30/20

(57) ABSTRACT

The present disclosure provides systems, devices, and methods for magnetic resonance imaging (MRI). The methods may include obtaining an intermediate file related to an MRI sequence. The intermediate file may be generated in a first development environment and be readable and convertible in a second development environment. The methods may include converting the intermediate file into one or more MRI sequence elements executable in the second development environment. The methods may include performing a verification operation on the one or more MRI sequence elements in the second development environment. The methods may further include, in response to a successful verification result of the verification operation, generating hardware instructions based on the one or more MRI sequence elements, and controlling an MRI device to execute the hardware instructions to apply the MRI sequence on the target subject.

### CROSS-REFERENCE TO RELATED APPLICATIONS

This application claims priority to Chinese Patent Application No. 202311538169.8, filed on Nov. 14, 2023, the contents of which are incorporated herein by reference.

### TECHNICAL FIELD

The present disclosure generally relates to the medical imaging field, and more particularly, relates to systems, devices, and methods for magnetic resonance imaging (MRI).

### BACKGROUND

Magnetic resonance (MR) technology is extensively utilized across various fields through diverse experimental methodologies. The primary distinction among these experimental methodologies lies in sequence timing diagrams employed. Typically, the development environment of each MR system is established based on its unique code framework, hardware modules, implementation logic, etc., which vary by manufacturer. This results in incompatible and non-transferable development environments, causing users to face significantly different development environments when using MR systems from different manufacturers. Consequently, high entry barriers in programming languages and development modes restrict users' ability to expand and explore MR applications and hamper the broader adoption and advancement of the MR technology.

Therefore, it is desirable to provide systems, devices, and methods for MRI, which facilitate MRI development by reducing the complexity of MR application development and enhancing development efficiency.

### SUMMARY

According to an aspect of the present disclosure, a method for magnetic resonance imaging (MRI) is provided. The method may be implemented on a computing device having at least one processor and at least one storage device. The method may include obtaining an intermediate file related to an MRI sequence. The intermediate file may be generated in a first development environment, and be readable and convertible in a second development environment. The method may include converting the intermediate file into one or more MRI sequence elements executable in the second development environment. The method may include performing, in the second development environment, a verification operation on the one or more MRI sequence elements. The method may further include, in response to a successful verification result of the verification operation, generating hardware instructions based on the one or more MRI sequence elements, and controlling an MRI device to execute the hardware instructions to apply the MRI sequence on a target subject.

In some embodiments, the second development environment may be a development environment of the MRI device, and the second development environment may be different from the first development environment.

In some embodiments, the intermediate file may include gradient information, radio frequency information, and analog-to-digital converter (ADC) information of the MRI sequence.

In some embodiments, the intermediate file may be converted from a first sequence file, the first sequence file may be generated in the first development environment.

In some embodiments, the first sequence file may include first values of one or more first parameters related to the MRI sequence, the one or more first parameters may include at least one of: a gradient parameter, a radio frequency parameter, and an ADC parameter of the MRI sequence. The first value of at least one of the one or more first parameters may be determined based on second values of one or more second parameters related to an MR scan to be performed based on the MRI sequence. The second values of the one or more second parameters may be input by a user in the first development environment, and the one or more second parameters may include at least one of: a parameter related to a field of view (FOV), a parameter related to a resolution, and a parameter related to a scan time of the MR scan.

In some embodiments, the methods may include obtaining updated information of the one or more second parameters input by the user in the first development environment; and updating the intermediate file by updating the first values of the one or more first parameters included in the intermediate file based on the updated information of the one or more second parameters.

In some embodiments, the intermediate file may be converted from the first sequence according a process including: determining a type of the first development environment; determining, from a plurality of scripts corresponding to different development environments, a target script corresponding to the type of the first development environment; and converting the first sequence file into the intermediate file based on the target script.

In some embodiments, the converting the intermediate file into one or more MRI sequence elements executable by the second development environment may include determining a type of the intermediate file; determining, from a plurality of interfaces corresponding to different file types, a target interface corresponding to the type of the intermediate file; and converting the intermediate file into the one or more MRI sequence elements using the target interface.

In some embodiments, one or more MRI sequence elements executable by the second development environment may include at least one of a data acquisition element, a gradient element, and a radio frequency element.

In some embodiments, the performing, in the second development environment, a verification operation on the one or more MRI sequence elements may include determining, in the second development environment, whether the one or more MRI sequence elements are implementable by the hardware of the MRI device.

In some embodiments, the performing, in the second development environment, a verification operation on the one or more MRI sequence elements may include generating, based on the one or more MRI sequence elements, a second sequence file; displaying the second sequence file to a user via a user terminal; obtaining feedback information with respect to the second sequence file input by the user via the user terminal; and generating, based on the feedback information, a verification result of the verification operation.

In some embodiments, the methods may include obtaining updated information related to the MRI sequence in the first development environment; and updating, based on the update information, the hardware instructions directly in the second development environment.

In some embodiments, the intermediate file includes template nodes for filling different types of information of the MRI sequence.

In some embodiments, the intermediate file is converted from a first sequence file, the first sequence file being generated in a third development environment different from the first development environment and the second development environment.

In some embodiments, parameters of one or more MRI sequence elements are determined based on parameters included in the intermediate file.

According to another aspect of the present disclosure, a system for MRI is provided. The system may include at least one storage device including a set of instructions; and at least one processor configured to communicate with the at least one storage device. When executing the set of instructions, the at least one processor may be configured to direct the system to perform operations. The operations may include obtaining an intermediate file related to an MRI sequence. The intermediate file may be generated in a first development environment, and be readable and convertible in a second development environment. The operations may include converting the intermediate file into one or more MRI sequence elements executable in the second development environment. The operations may include performing, in the second development environment, a verification operation on the one or more MRI sequence elements. The operations may further include, in response to a successful verification result of the verification operation, generating hardware instructions based on the one or more MRI sequence elements, and controlling an MRI device to execute the hardware instructions to apply the MRI sequence on a target subject.

According to another aspect of the present disclosure, a non-transitory computer readable medium is provided. The non-transitory computer readable medium may include executable instructions that, when executed by at least one processor, direct the at least one processor to perform a method. The method may include obtaining an intermediate file related to an MRI sequence. The intermediate file may be generated in a first development environment, and be readable and convertible in a second development environment. The method may include converting the intermediate file into one or more MRI sequence elements executable in the second development environment. The method may include performing, in the second development environment, a verification operation on the one or more MRI sequence elements. The method may further include, in response to a successful verification result of the verification operation, generating hardware instructions based on the one or more MRI sequence elements, and controlling an MRI device to execute the hardware instructions to apply the MRI sequence on a target subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2A is a block diagram illustrating an exemplary processing device according to some embodiments of the present disclosure;

FIG. 2B is a block diagram illustrating an exemplary processing device according to some embodiments of the present disclosure;

FIG. 3 is a schematic diagram illustrating an exemplary process for MRI according to some embodiments of the present disclosure;

FIG. 4A is a schematic diagram illustrating an exemplary first sequence file according to some embodiments of the present disclosure;

FIG. 4B is a schematic diagram illustrating an exemplary second sequence file according to some embodiments of the present disclosure;

FIG. 5 is a schematic diagram illustrating an exemplary process for generating a first sequence file according to some embodiments of the present disclosure;

FIG. 6 is a schematic diagram illustrating an exemplary process for generating an intermediate file according to some embodiments of the present disclosure;

FIG. 7 is a schematic diagram illustrating an exemplary process for generating one or more MRI sequence elements according to some embodiments of the present disclosure;

FIG. 8 is a schematic diagram illustrating an exemplary process for transmitting an intermediate file from a first development environment to a second development environment according to some embodiments of the present disclosure;

FIG. 9 is a schematic diagram illustrating an exemplary process for MRI according to some embodiments of the present disclosure; and

FIG. 10 is a schematic diagram illustrating an exemplary imaging device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for non-invasive biomedical imaging/treatment, such as for disease diagnosis, disease therapy, or research purposes. In some embodiments, the systems may include an imaging system. The imaging system may include a single modality system and/or a multi-modality system. The term “modality” used herein broadly refers to an imaging or treatment method or technology that gathers, generates, processes, and/or analyzes imaging information of a subject or treatments the target subject. The single modality system may include, for example, an ultrasound imaging system, an X-ray imaging system, a computed tomography (CT) system, a magnetic resonance imaging (MRI) system, an ultrasonography system, a positron emission tomography (PET) system, an optical coherence tomography (OCT) imaging system, an ultrasound (US) imaging system, an intravascular ultrasound (IVUS) imaging system, a near-infrared spectroscopy (NIRS) imaging system, a digital subtraction angiography (DSA) system, or the like, or any combination thereof. The multi-modality system may include, for example, an X-ray imaging-magnetic resonance imaging (X-ray-MRI) system, a positron emission tomography-X-ray imaging (PET-X-ray) system, a single photon emission computed tomography-magnetic resonance imaging (SPECT-MRI) system, a positron emission tomography-computed tomography (PET-CT) system, a C-arm system, a positron emission tomography-magnetic resonance imaging (PET-MRI) system, a digital subtraction angiography-magnetic resonance imaging (DSA-MRI) system, etc. It should be noted that the medical system described below is merely provided for illustration purposes, and is not intended to limit the scope of the present disclosure.

In the present disclosure, the target subject may include a biological object and/or a non-biological object. The biological object may be a human being, an animal, a plant, or a specific portion, organ, and/or tissue thereof. For example, the target subject may include the head, the neck, the thorax, the heart, the stomach, a blood vessel, a soft tissue, a tumor, a nodule, or the like, or any combination thereof. In some embodiments, the target subject may be a man-made composition of organic and/or inorganic matters that are with or without life. The terms “object” and “subject” are used interchangeably in the present disclosure.

In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiments, the image may be a medical image, an optical image, etc.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the target subject, or a portion thereof, or an image including the target subject, or a portion thereof, for brevity. Still further, an operation performed on a representation of a subject, or a portion thereof, in an image may be referred to as an operation performed on the target subject, or a portion thereof, for brevity. For instance, a segmentation of a portion of an image including a representation of an ROI from the image may be referred to as a segmentation of the ROI for brevity.

A medical imaging system (e.g., an MRI system) may include a user interface that enables a user to obtain or view images, a data acquisition device that acquires data from a target subject using a certain imaging modality, an image reconstruction processor that reconstructs an image using the acquired data, and a storage device for storing the image and related information of the target subject. Generally, the hardware of the medical imaging system is designed to implement the above functions, and system software (e.g., execution instructions corresponding to a sequence timing diagram) is designed and written for the hardware.

Typically, the development environment of an MR system is established based on the unique code framework, hardware modules, implementation logic, etc., specific to the manufacturer of the MR system. This results in that development environments of MR systems corresponding to different manufacturers are incompatible and non-transferable. Consequently, users face significantly different development environments when using MR systems from different manufacturers, leading to high entry barriers in programming languages and development modes. When the user needs to design an MRI sequence, he or she needs to learn knowledge about the code framework, the hardware modules, the implementation logic, etc., at first. Alternatively, the user needs the support of the manufacturer's engineers, which restricts users' ability to expand and explore MR applications and hampers the broader adoption and advancement of the MR technology.

The present disclosure relates to systems, devices, and methods for MRI. The methods may include obtaining an intermediate file related to an MRI sequence. The intermediate file may be generated in a first development environment and be readable and convertible in a second development environment. The methods may include converting the intermediate file into one or more MRI sequence elements executable in the second development environment. The methods may include performing a verification operation on the one or more MRI sequence elements in the second development environment. The methods may further include, in response to a successful verification result of the verification operation, generating hardware instructions based on the one or more MRI sequence elements, and controlling an MRI device to execute the hardware instructions to apply the MRI sequence on the target subject.

By introducing the intermediate file, the one or more MRI sequence elements and the hardware instructions can be generated in the second development environment, even if the intermediate file is generated in the first development environment with distinct development language and development mode from the second development environment. This approach eliminates incompatibility between the first development environment and the second development environment, thereby reducing the complexity of MR application development and enhancing development efficiency. In addition, the user can design the intermediate file in the first development environment, which can reduce learning and time cost associated with the second development environment, thereby further boosting the development efficiency.

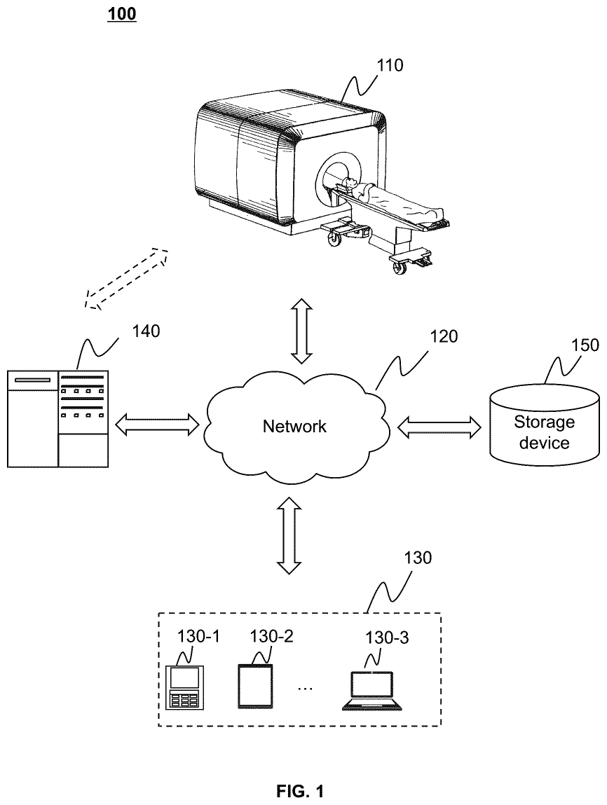

FIG. 1 is a schematic diagram illustrating an exemplary imaging system 100 according to some embodiments of the present disclosure. As shown in FIG. 1, the imaging system 100 may include an imaging device 110, a network 120, one or more terminals 130, a processing device 140, and a storage device 150. In some embodiments, the imaging device 110, the terminal device 130, the processing device 140, and/or the storage device 150 may be connected to and/or communicate with each other via a wireless connection, a wired connection, or a combination thereof. The connection among the components of the imaging system 100 may be variable. Merely by way of example, the imaging device 110 may be connected to the processing device 140 through the network 120, as illustrated in FIG. 1. As another example, the imaging device 110 may be connected to the processing device 140 directly. As a further example, the storage device 150 may be connected to the processing device 140 through the network 120, as illustrated in FIG. 1, or connected to the processing device 140 directly.

The imaging device 110 may be configured to generate or provide image data by scanning a target subject (or at least a portion of the target subject). In some embodiments, the imaging device 110 may include a single modality imaging device or a multi-modality imaging device.

Merely by way of example, the imaging device 110 may be an MRI device. The MRI device may be configured to scan the target subject (or a portion of the target subject) to acquire scan data, such as magnetic resonance (MR) signals associated with the target subject. For example, the MRI device may detect MR signals by executing hardware instructions to apply an MRI sequence on the target subject.

In some embodiments, the MRI device may include, for example, a magnetic body, a gradient coil assembly, a radiofrequency (RF) coil assembly, etc. In some embodiments, the MRI device may be a permanent magnet MR scanner, a superconducting electromagnet MR scanner, a resistive electromagnet MR scanner, etc., according to types of the magnetic body. In some embodiments, the MRI device may be a high-field MR scanner, a mid-field MR scanner, a low-field MR scanner, etc., according to the intensity of the magnetic field.

The magnetic body may generate a first magnetic field (also referred to as a main magnetic field) for polarizing the target subject to be scanned. The gradient coil assembly may generate a second magnetic field (also referred to as a gradient magnetic field). The gradient coil assembly may include X-gradient coils, Y-gradient coils, and Z-gradient coils. The gradient coil assembly may generate one or more magnetic field gradient pulses to the main magnetic field in an X direction (Gx), a Y direction (Gy), and a Z direction (Gz) to encode spatial information of the target subject. The RF coil assembly may include a plurality of RF coils. The RF coils may include one or more RF transmit coils and/or one or more RF receiver coils. The RF transmit coil(s) may transmit RF pulses to the target subject. Under the coordinated action of the main magnetic field, the gradient magnetic field, and the RF pulses, MR signals relating to the target subject may be generated according to a pulse sequence. The RF receiver coils may acquire the MR signals from the target subject according to the pulse sequence. The MR signals may be processed using a transform operation (e.g., Fourier Transform) to fill a k-space to obtain k-space data. The k-space data may be reconstructed according to an MR reconstruction algorithm (e.g., a back projection technique, an iteration reconstruction technique) to obtain an MR image of the target subject.

In some embodiments, the MRI device may further include an analog-to-digital converter (ADC). The ADC may be configured to convert the MR signals received by the one or more RF receiver coils into MRI data for generating the MR image. Exemplary ADCs may include a direct conversion ADC, a successive approximation ADC, a ramp comparison ADC, a Wilkinson ADC, am integral ADC, an incremental coding ADC, a pipeline ADC, a sigma-incremental ADC, or the like, or any combination thereof.

The network 120 may include any suitable network that can facilitate the exchange of information and/or data for the imaging system 100. In some embodiments, one or more components (e.g., the imaging device 110, the terminal 130, the processing device 140, the storage device 150, etc.) of the imaging system 100 may communicate information and/or data with one or more other components of the imaging system 100 via the network 120. For example, the processing device 140 may obtain image data from the imaging device 110 via the network 120. As another example, the processing device 140 may obtain user instructions from the terminal 130 via the network 120. In some embodiments, the network 120 may include one or more network access points.

The terminal(s) 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the terminal(s) 130 may include a processing unit, a display unit, a sensing unit, an input/output (I/O) unit, a storage unit, etc. In some embodiments, the display unit may include an interactive interface that is configured to receive an input from a user. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, the processing device 140 may obtain an intermediate file related to an MRI sequence, and convert the intermediate file into one or more MRI sequence elements executable in a second development environment. As another example, the processing device 140 may perform a verification operation on the one or more MRI sequence elements in the second development environment. As still another example, in response to a successful verification result of the verification operation, the processing device 140 may generate hardware instructions based on the one or more MRI sequence elements, and control the imaging device 110 (e.g., the MRI device) to execute the hardware instructions to apply the MRI sequence on the target subject. In some embodiments, the processing device 140 may be a single server or a server group. The server group may be centralized or distributed. In some embodiments, the processing device 140 may be local or remote. In some embodiments, the processing device 140 may be implemented on a cloud platform.

In some embodiments, the processing device 140 may be implemented by a computing device. For example, the computing device may include a processor, a storage, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

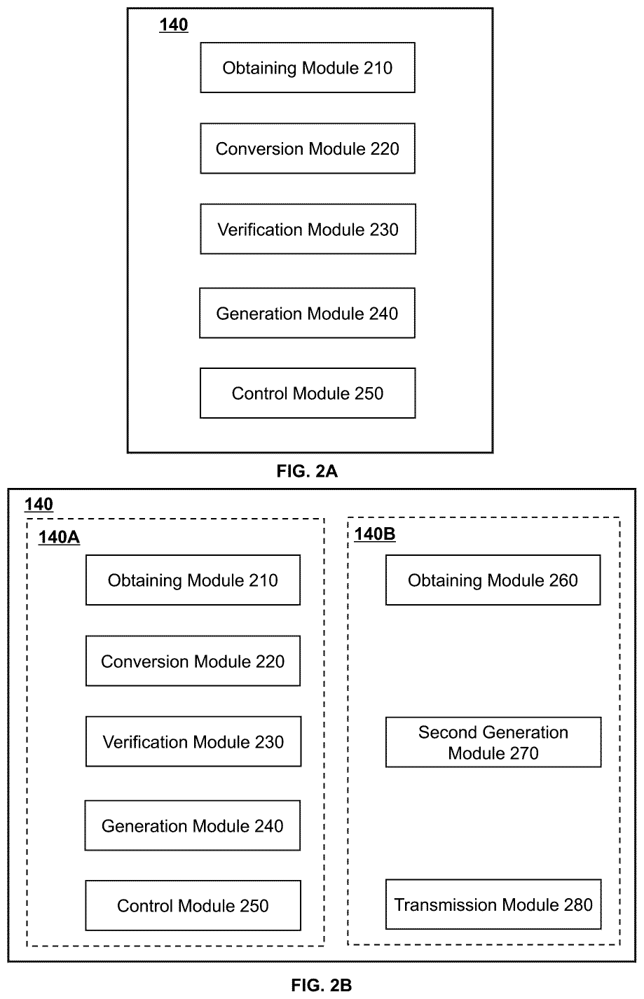

FIG. 2A is a block diagram illustrating an exemplary processing device 140 according to some embodiments of the present disclosure. The modules illustrated in FIG. 2A may be implemented on the processing device 140. In some embodiments, the processing device 140 may be in communication with a computer-readable storage medium (e.g., the storage device 150 illustrated in FIG. 1) and execute instructions stored in the computer-readable storage medium. The processing device 140 may include an obtaining module 210, a conversion module 220, a verification module 230, a generation module 240, and a control module 250.

The obtaining module 210 may be configured to obtain an intermediate file related to an MRI sequence. The intermediate file may be generated in a first development environment and be readable and convertible in a second development environment. More descriptions regarding the obtaining the intermediate file may be found elsewhere in the present disclosure. See, e.g., operation 302 and relevant descriptions thereof.

The conversion module 220 may be configured to convert the intermediate file into one or more MRI sequence elements executable in the second development environment. More descriptions regarding the conversion of the intermediate file may be found elsewhere in the present disclosure. See, e.g., operation 304 and relevant descriptions thereof.

The verification module 230 may be configured to perform a verification operation on the one or more MRI sequence elements in the second development environment. More descriptions regarding the verification operation may be found elsewhere in the present disclosure. See, e.g., operation 306 and relevant descriptions thereof.

The generation module 240 may be configured to generate hardware instructions based on the one or more MRI sequence elements in response to a successful verification result of the verification operation. More descriptions regarding the generation of the hardware instructions may be found elsewhere in the present disclosure. See, e.g., operation 308 and relevant descriptions thereof.

The control module 250 may be configured to control an MRI device (e.g., the imaging device 110) to execute the hardware instructions to apply the MRI sequence on a target subject. More descriptions regarding the control of the MRI device may be found elsewhere in the present disclosure. See, e.g., operation 310 and relevant descriptions thereof.

It should be noted that the above descriptions of the processing device 140 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units.

In some embodiments, the processing device 140 may include a first portion operated in the second development environment and a second portion operated in the first development environment. Merely by way of example, referring to FIG. 2B, FIG. 2B is a block diagram illustrating an exemplary processing device 140 according to some embodiments of the present disclosure.

As illustrated in FIG. 2B, the processing device 140 may include a first portion 140A and a second portion 140B, and the first portion 140A may be in communication with the second portion 140B. Modules in the first portion 140A may be operated in a second development environment, and modules in the second portion 140B may be operated in a first development environment.

The modules in the first portion 140A may be similar to the modules described in FIG. 2A.

The modules in the second portion 140B may include an obtaining module 260, a generation module 270, and a transmission module 280.

The obtaining module 260 may be configured to obtain value(s) of one or more parameters related to an MRI sequence. The one or more parameters may include at least one first parameter. The at least one first parameter may include a gradient parameter, a radio frequency parameter, an ADC parameter, or the like, or any combination thereof. In some embodiments, the one or more parameters may further include at least one second parameter. The at least one first parameter may include a parameter related to a field of view (FOV), a parameter related to a resolution, and a parameter related to a scan time, or the like, or any combination thereof, of an MR scan to be performed based on the MRI sequence. Correspondingly, the value(s) may include at least one first value and/or at least one second value. More descriptions regarding the obtaining the intermediate file may be found elsewhere in the present disclosure. See, e.g., operation 802 and relevant descriptions thereof.

The generation module 270 may be configured to generate an intermediate file based on the value(s) of the one or more parameters in a first development environment. The intermediate file may be generated in the first development environment and be readable and convertible in a second development environment. More descriptions regarding the generation of the intermediate file may be found elsewhere in the present disclosure. See, e.g., operation 804 and relevant descriptions thereof.

The transmission module 280 may be configured to transmit the intermediate file to the second development environment. More descriptions regarding the transmission of the intermediate file may be found elsewhere in the present disclosure. See, e.g., operation 806 and relevant descriptions thereof.

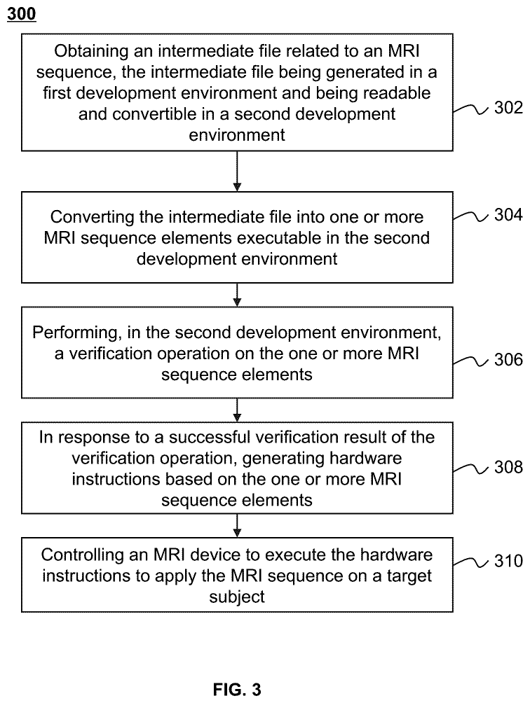

FIG. 3 is a flowchart illustrating an exemplary process 300 for magnetic resonance imaging according to some embodiments of the present disclosure.

In 302, the processing device 140 (e.g., the obtaining module 210) may obtain an intermediate file related to an MRI sequence. The intermediate file may be generated in a first development environment and be readable and convertible in a second development environment.

The MRI sequence refers to a specific set of instructions, operations, settings, and parameters for an MRI device (e.g., an MRI scanner, the imaging device 110) to use for acquiring MRI data of a target subject during an MR scan. Each MRI sequence uses different instructions, operations, settings, and parameters to emphasize certain properties of tissues, producing varying types of MRI data. For example, the MRI sequence may include a plurality of MRI sequence elements (or referred to as MRI sequence modules) and parameters corresponding to each of the plurality of MRI sequence elements. The plurality of MRI sequence elements may include a gradient element, an RF element, a data acquisition element, or the like, or any combination thereof. Exemplary MRI sequences may include a T1-weighted imaging (T1WI) sequence, a T2-weighted imaging (T2WI) sequence, a fluid-attenuated inversion recovery (FLAIR) sequence, a diffusion-weighted imaging (DWI) sequence, a susceptibility-weighted imaging (SWI) sequence, a short tau inversion recovery (STIR) sequence, a magnetic resonance angiography (MRA) sequence, a gradient echo (GRE) sequence, a spin-echo sequence, or the like, or any combination thereof. As another example, the MRI sequence may be a customized MRI sequence. For instance, the MRI sequence may be customized for the target subject based on information (e.g., an age, a gender, a target region, a medical history, a doctor's advice, etc.) of the target subject.

The intermediate file refers to a file that includes information relating to the MRI sequence (e.g., the instructions, operations, settings, and parameters of the MRI sequence).

In some embodiments, the intermediate file may include gradient information, radio frequency (RF) information, analog-to-digital converter (ADC) information, or the like, or any combination thereof, of the MRI sequence.

The gradient information refers to gradient waveform information configured to control changes of gradient magnetic fields during the MR scan on the target subject. For example, the gradient information may reflect parameter information of the gradient element corresponding to the MRI sequence. Exemplary gradient information may include a gradient name (e.g., a readout gradient (RO gradient, also referred to as a frequency encoding gradient), a phase encoding gradient (PE gradient), a slice selection gradient (SS gradient), etc.), a coordinate axis to which a gradient is applied (e.g., an SS axis, a PE axis, an RO axis), a gradient amplitude, a gradient start time, a gradient climbing time, a gradient plateau time, a gradient descent time, a gradient size, a gradient switching time, or the like, or any combination thereof. For example, for a gradient 1, the gradient name is SS gradient, the gradient start time is 0 milliseconds (ms), the gradient climbing time is 0.2 ms, the gradient plateau time is 3 ms, the gradient descent time is 0.2 ms, and the gradient size is 20 milli teslas per meter (mT/m).

The RF information refers to information regarding an RF pulse sequence used to excite the target subject during the MR scan. For example, the RF information may reflect parameter information of the RF element corresponding to the MRI sequence. Exemplary RF information may include an RF name, an RF angle, an RF waveform, an RF amplitude, an RF start time, an RF duration, an RF center frequency, or the like, or any combination thereof. For example, for an RF pulse, the RF name is refocus pulse, the RF angle is 150 degree, the RF waveform is a sinc pulse of 4 tbw, the RF start time is 0.2 ms, and the RF duration is 3 ms.

The ADC information refers to information that converts analog signals to digital signals during the MR scan. For example, the ADC information may reflect parameter information of the data acquisition element corresponding to the MRI sequence. Exemplary ADC information may include an ADC name, an ADC start time, a sampling start time, a sampling interval, a sampling ratio, a number of sampling points, or the like, or any combination thereof. For example, for an ADC1, the ADC name is ADC, the ADC start time is 3.8 ms, the sampling interval is 1000 nanoseconds (ns), the number of sampling points is determined by a user (e.g., a doctor, a technician, etc.) based on actual needs (e.g., an imaging size, a voxel size, etc.).

In some embodiments, the intermediate file may further include supplementary information. For example, the supplementary information may include trigger information and/or circle information.

The trigger information refers to timing signals that help synchronize the initiation or timing of the MRI sequence with physiological events or external devices. For example, the trigger information may include an internal trigger (e.g., timing for RF pulses, gradient pulses, data acquisition, etc.), an external trigger (e.g., cardiac triggering, respiratory triggering/gating, an event-related trigger, etc.), a timing parameter (e.g., repetition time and echo time), or the like, or any combination thereof.

The circle information refers to a number of cycles of the MRI sequence (or a portion of the MRI sequence) during the MR scan. For example, the circle information of 5 may indicate that the MRI sequence needs to be executed five times during the MR scan. As another example, when the circle information is 1 or the intermediate file includes no circle information, the MRI sequence may be executed once during the MR scan.

A development environment refers to a set of tools, software, and configurations that are used to write, test, and debug code (e.g., the intermediate file). In some embodiments, the development environment may include a software development environment and an integrated development environment. The software development environment may be a set of software used to support engineering development and maintenance of system software and application software. The integrated development environment may be an application used to provide a program development environment, typically including a code editor, a compiler, a debugger, a graphical user interface tool, etc. In some embodiments, the integrated development environment may integrate functions including, such as, a code writing function, an analysis function, a compilation function, a debugging function, etc. In other words, any software or software suite (group) that integrates the above functions can be regarded as the integrated development environment. For example, the integrated development environment may include a MATLAB development environment, a Visual Studio development environment, a C++ development environment, a Delphi development environment, etc.

In some embodiments, the development environment may be established based on computer language, such as algorithm language, database language, intelligent simulation language, etc. Correspondingly, the development environment may include a Java development environment, a Python development environment, a Visual Studio development environment, a C++ development environment, a Delphi development environment, etc.

The first development environment refers to a development environment configured to develop the intermediate file. For example, the first development environment may be established in a terminal device (e.g., the terminal(s) 130). The second development environment may be a development environment of the MRI device. In some embodiments, the second development environment may be different from the first development environment. For example, the first development environment may be MATLAB or a development environment based on python, which is familiar to the user; while the second development environment may be a development environment designed for the MRI device, which is unfamiliar to the user.

In some embodiments, the computer language of the first development environment may be the same as or different from that of the second development environment. For example, the first development environment and the second development environment may correspond to C++ language. As another example, the first development environment may correspond to C++ language, and the second development environment may correspond to python language.

In some embodiments, a file format of the intermediate file may be any form that can be readable and convertible in the second development environment. For example, the file format of the intermediate file may include an extensible markup language (XML) format, a text format, a binary format, a key-value pair format, or the like, or any combination thereof.

In some embodiments, the file format of the intermediate file may be configured such that the information (e.g., the gradient information, the RF information, the ADC information, the supplementary information, etc.) corresponding to the MRI sequence can be read in the second development environment. In some embodiments, the intermediate file includes template nodes for filling different types of information of the MRI sequence. Merely by way of example, taking the intermediate file in the text form as an example, the intermediate file may include a plurality of template nodes configured to fill the information corresponding to the MRI sequence. For example, the plurality of template nodes may include a circle template node used to fill the circle information and a block template node used to fill the gradient information (e.g., gradient parameters), the RF information (e.g., RF parameters), the ADC information (e.g., ADC parameters), etc. As another example, the plurality of template nodes may include a gradient template node used to fill the gradient information, an RF template node used to fill the RF information, an ADC template node used to fill the ADC information, and a supplementary template node used to fill the supplementary information (e.g., the trigger information and/or the circle information). For instance, taking the gradient template node as an example, the gradient information in the intermediate file may be expressed as “<Grad Trap>SS_RefSliceSelection: 20_0_0.2_3_0.2</Grad Trap>,” wherein “Grad Trap” indicates a gradient element, “SS” indicates that the gradient acts on the SS axis, “RefSliceSelection” is the gradient name, and 20_0_0.2_3_0.2 indicates that the gradient amplitude is 20 mT/m, the gradient start time is 0 ms, the gradient climbing time is 0.2 ms, the gradient plateau time is 3 ms, and the gradient descent time is 0.2 ms.

In some embodiments, the processing device 140 may generate the intermediate file based on a user input. For example, the user may input the information of the MRI sequence through a user interface corresponding to the first development environment, and the processing device 140 may generate the intermediate file by filling the plurality of template nodes in the intermediate file based on the input information of the MRI sequence.

In some embodiments, the processing device 140 may generate the intermediate file based on a first sequence file.

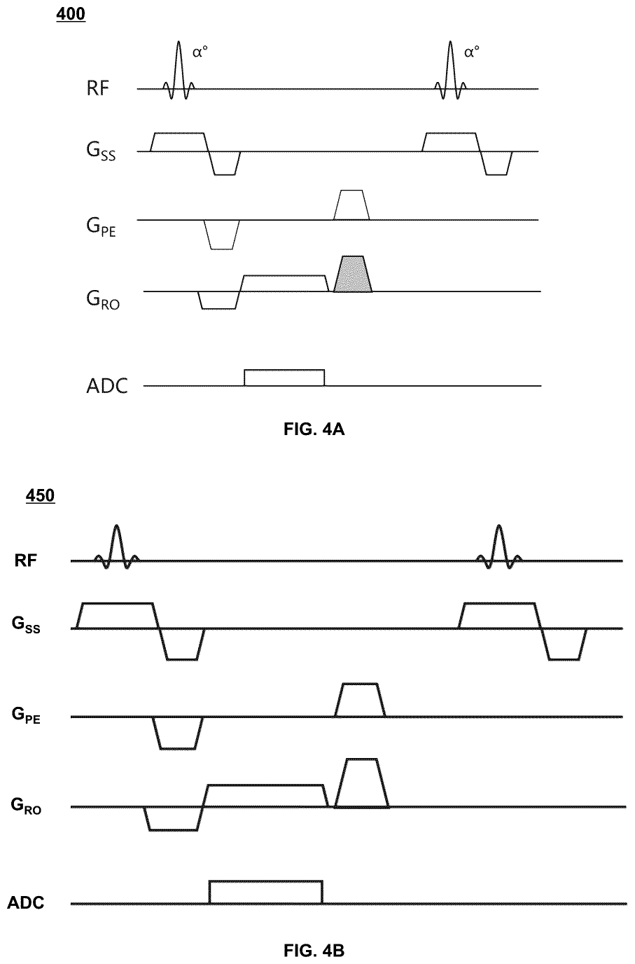

The first sequence file refers to a waveform file of the MRI sequence in the first development environment. The first sequence file may include first values of one or more first parameters related to the MRI sequence. The one or more first parameters may include a gradient parameter, an RF parameter, an ADC parameter of the MRI sequence, or the like, or any combination thereof. Merely by way of example, referring to FIG. 4A, FIG. 4A is a schematic diagram illustrating an exemplary first sequence file 400 according to some embodiments of the present disclosure. As illustrated in FIG. 4A, the first sequence file 400 may include the RF parameter (a waveform corresponding to “RF”), the gradient parameter (waveforms corresponding to “G_{SS},” “G_{PE},” and “G_{RO}”), and the ADC parameter (a waveform corresponding to “ADC”).

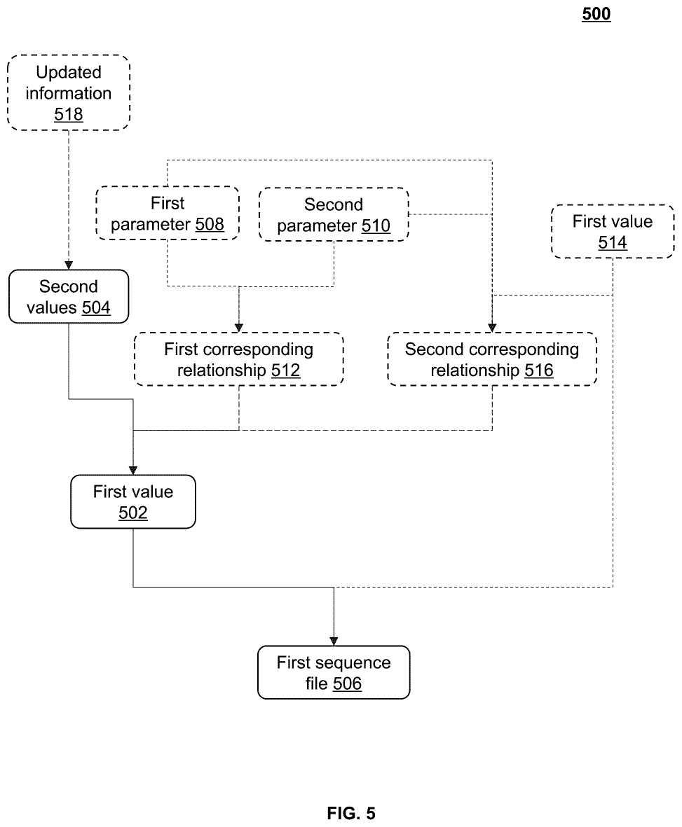

In some embodiments, the first value of at least one of the one or more first parameters may be determined based on second values of one or more second parameters related to the MR scan to be performed based on the MRI sequence. The one or more second parameters may include a parameter related to a field of view (FOV) or a region of interest (ROI), a parameter related to a resolution, a parameter related to a scan time of the MR scan. More descriptions regarding the determination of the first value of at least one of the one or more first parameters may be found elsewhere in the present disclosure. See, e.g., FIG. 5 and relevant descriptions thereof.

In some embodiments, the first sequence file may be generated in the first development environment. For example, the first sequence file may be generated in the first development environment through the computer language of the first development environment based on the information of the target subject. In some embodiments, a file format of the first sequence file may be any form, such as, an image format, a text format, etc.

In some embodiments, the intermediate file may include the first sequence file. For example, if the file format of the first sequence file is consistent with the file format of the intermediate file (i.e., the information of the MRI sequence in the first sequence file can be read in the second development environment), the processing device 140 may designate the first sequence file as the intermediate file. Merely by way of example, if the first development environment is MATLAB, the user may generate the first sequence file including five arrays through the MATLAB, wherein the five arrays may include a first array corresponding to the RF element, a second array corresponding to the SS gradient element, a third array corresponding to the PE gradient element, a fourth array corresponding to the RO gradient element, and a fifth array corresponding to the data acquisition element. Each value in the first array may indicate an amplitude of a point in RF pulses. Each value in the second array may indicate an amplitude of a point in the SS gradient. Each value in the third array may indicate an amplitude of a point in the PE gradient. Each value in the fourth array may indicate an amplitude of a point in the RO gradient. Each value in the fifth array may indicate a sampling time during the MR scan. Therefore, the information of the MRI sequence can be read from the first sequence file in the second development environment. At this time, the first sequence file may be regarded as the intermediate file.

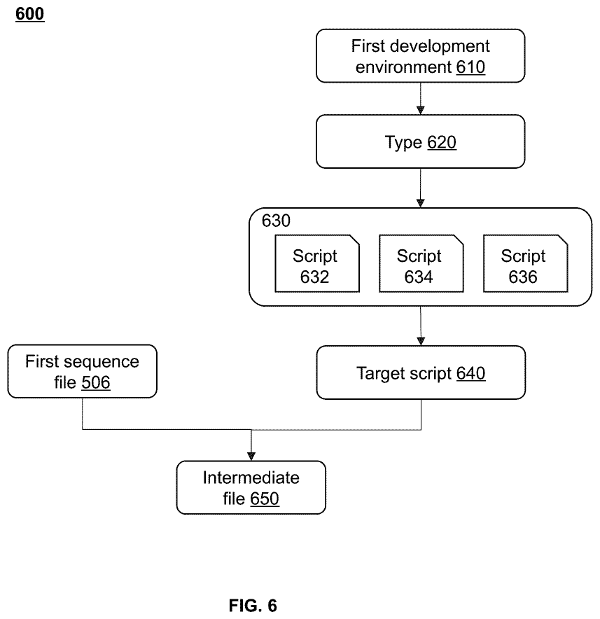

In some embodiments, the intermediate file may be converted from the first sequence file. For example, the user may fill the plurality of template nodes in the intermediate file based on the first sequence file. In some embodiments, the intermediate file may be automatically converted from the first sequence file using a script. For example, the processing device 140 may convert the first sequence file that includes the information (e.g., the gradient information, the RF information, the ADC information, the supplementary information, etc.) corresponding to the MRI sequence and is generated in the first development environment into the intermediate file through a script corresponding to the first development environment. For instance, the processing device 140 may determine a type of the first development environment, determine a target script corresponding to the type of the first development environment from a plurality of scripts corresponding to different development environments, and convert the first sequence file into the intermediate file based on the target script. More descriptions regarding the converting the first sequence file into the intermediate file may be found elsewhere in the present disclosure. See, e.g., FIG. 6 and relevant descriptions thereof.

In some embodiments, the processing device 140 may obtain the intermediate file through a cloud disk, a network disk, a local disk, a mobile disk, TCP/IP protocol, or the like, or any combination thereof. For example, after the intermediate file is generated, the intermediate file may be copied to a mobile disk, and the mobile disk may be connected to the imaging system (e.g., the MRI device). The processing device 140 may retrieve the intermediate file from the mobile disk.

In some embodiments, a storing location of the intermediate file may be prespecified. For example, the user may pre-set to store the intermediate file in the cloud disk, and the processing device 140 of the imaging system 100 may read the intermediate file from the cloud disk.

In some embodiments, the first sequence file is generated in a third development environment different from the first development environment and the second development environment. For example, the first sequence file is generated in a Python development environment, the intermediate file is generated in a MATLAB development environment, and the second development environment is a C++ development environment.

In 304, the processing device 140 (e.g., the conversion module 220) may convert the intermediate file into one or more MRI sequence elements executable in the second development environment.

The one or more MRI sequence elements may include the gradient element, the RF element, the data acquisition element (ADC element), or the like, or any combination thereof.

In some embodiments, the processing device 140 may obtain the information of the MRI sequence by parsing the intermediate file, and convert the information of the MRI sequence into the one or more MRI sequence elements executable in the second development environment. For example, in combination with the above description, the processing device 140 may read the intermediate file in the second development environment. When the circle template node is read, the processing device 140 may parse contents in the circle template node through an assemble circle function developed by the second development environment to obtain the circle information. Then, when the block template node is read, the processing device 140 may parse contents in the block template node through an assemble task block function developed in the second development environment to obtain the gradient information, the RF information, the ADC information, etc. Finally, the processing device 140 may set the one or more MRI sequence elements (e.g., the gradient element, the RF element, the data acquisition element, etc.) based on the parsed contents (including the circle information, the gradient information, the RF information, the ADC information, etc.).

In some embodiments, the intermediate file includes parameters of the MRI sequences (e.g., gradient parameters, RF parameters, ADC parameters), and the processing device 140 may determine parameters of the MRI sequence elements based on the parameters included in the intermediate file. For example, as described above, the intermediate file includes template nodes corresponding to the gradient information, the RF information, and the ADC information, the processing device 140 may determine the gradient parameters, the RF parameters, and the ADC parameters based on the corresponding template nodes.

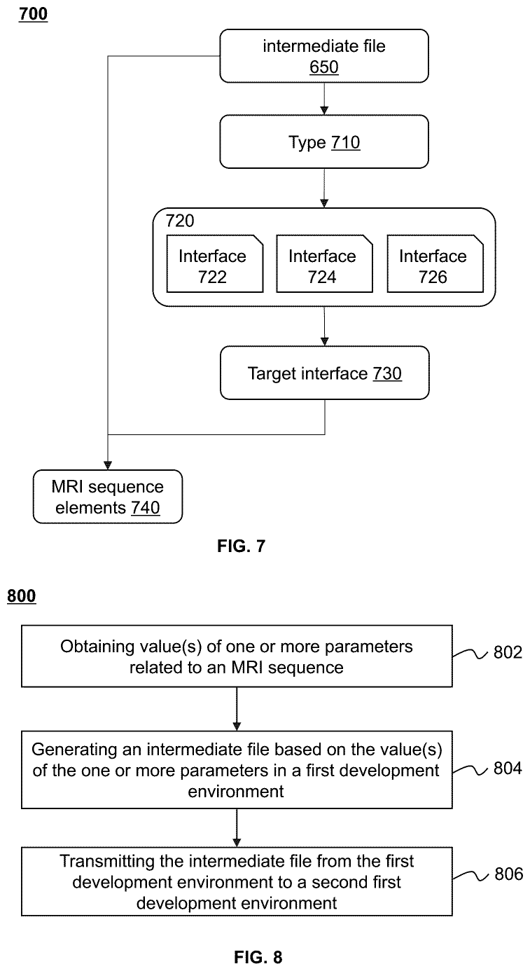

In some embodiments, the processing device 140 may convert the intermediate file into the one or more MRI sequence elements based on the type of the intermediate file. For example, the processing device 140 may determine the type of the intermediate file, and determine a target interface corresponding to the type of the intermediate file from a plurality of interfaces corresponding to different file types. Further, the processing device 140 may convert the intermediate file into the one or more MRI sequence elements using the target interface. More descriptions regarding the converting the intermediate file into the one or more MRI sequence elements may be found elsewhere in the present disclosure. See, e.g., FIG. 7 and relevant descriptions thereof.

In 306, the processing device 140 (e.g., the verification module 230) may perform a verification operation on the one or more MRI sequence elements in the second development environment.

The verification operation may be configured to determine whether the one or more MRI sequence elements can be executed successfully in the second development environment or an MRI device (e.g., the imaging device 110).

In some embodiments, the verification operation may include a compile validation, an operation validation, a parameter validation, a consistency validation, or the like, or any combination thereof.

The compile validation refers to verifying whether compile vulnerabilities exist in the code corresponding to the one or more MRI sequence elements. For example, the processing device 140 may perform the compile validation by checking for grammatical errors, logical errors, etc., in the code corresponding to the one or more MRI sequence elements.

The operation validation refers to verifying whether code corresponding to the one or more MRI sequence elements can be executed properly. For example, the processing device 140 may determine whether the one or more MRI sequence elements are implementable by the hardware of the MRI device in the second development environment. If the one or more MRI sequence elements are implementable, the processing device 140 may generate a successful verification result of the verification operation (e.g., a verification result indicating that the operation validation is passed). If the one or more MRI sequence elements are not implementable, the processing device 140 may generate a failing verification result of the verification operation (e.g., a verification result indicating that the operation validation is failed).

The parameter validation refers to verifying whether a third value of each third parameter corresponding to the one or more MRI sequence elements is within a corresponding value range. For example, for each third parameter, the processing device 140 may determine a value range corresponding to the third parameter, and perform the parameter validation on the third parameter by comparing the third value with the value range. If the third value is within the value range, the processing device 140 may generate a successful verification result of the verification operation (e.g., a verification result indicating that the parameter validation is passed). If the third value exceeds the value range, the processing device 140 may generate a failing verification result of the verification operation (e.g., a verification result indicating that the parameter validation is failed). In some embodiments, the value range may be determined based on system settings or set manually. For example, the processing device 140 may determine the value range based on configurations of the MRI device (e.g., the imaging device 110).

The consistency validation refers to verifying whether information of the one or more MRI sequence elements is consistent with the information in the intermediate file. For example, the processing device 140 may generate a second sequence file based on the one or more MRI sequence elements, and perform the consistency validation based on the second sequence file. The second sequence file refers to a waveform file of the MRI sequence generated in the second development environment based on the one or more MRI sequence elements. In some embodiments, the second sequence file may be similar to the first sequence file. Merely by way of example, referring to FIG. 4B, FIG. 4B is a schematic diagram illustrating an exemplary second sequence file 450 according to some embodiments of the present disclosure. As illustrated in FIG. 4B, the second sequence file 450 may include an RF parameter (a waveform corresponding to “RF”), a gradient parameter (waveforms corresponding to “G_{SS},” “G_{PE},” and “G_{RO}”), and an ADC parameter (a waveform corresponding to “ADC”).

In some embodiments, the processing device 140 may display the second sequence file to a user via a user terminal, obtain feedback information with respect to the second sequence file input by the user via the user terminal, and generate a verification result of the verification operation (e.g., the consistency validation) based on the feedback information. The user may be a user of the first development environment (e.g., the user who inputs the intermediate file) or another user. The feedback information may include feedback of the user regarding whether the information in the one or more MRI sequence elements is right and/or whether the information in the one or more MRI sequence elements is consistent with the information in the intermediate file. For example, the feedback information may include words, numbers, letters, etc. For instance, the feedback information may include a word “True” (or a number “1,” a letter “T”) indicating that the information in the one or more MRI sequence elements is right and/or the information in the one or more MRI sequence elements is consistent with the information in the intermediate file, and a word “False” (or a number “0,” a letter “F”) indicating that the information in the one or more MRI sequence elements is wrong and/or the information in the one or more MRI sequence elements is inconsistent with the information in the intermediate file. Correspondingly, the processing device 140 may generate a successful verification result of the verification operation (e.g., a verification result indicating that the consistency validation is passed) when the feedback information includes the word “True” (or the number “1,” the letter “T”). Alternatively, the processing device 140 may generate a failing verification result of the verification operation (e.g., a verification result indicating that the consistency validation is failed) when the feedback information includes the word “False” (or the number “0,” the letter “F”).

Merely by way of example, the processing device 140 may display the second sequence file 450 to the user through the user interface of the terminal(s) 130. When the user inputs “True” through the user interface, the processing device 140 may generate the successful verification result of the verification operation. When the user inputs “False” through the user interface, the processing device 140 may generate the failing verification result of the verification operation.

In some embodiments, the processing device 140 may generate the verification result of the verification operation (e.g., the consistency validation) based on the first sequence file and the second sequence file. For example, the processing device 140 may compare the first sequence file and the second sequence file using an image comparison algorithm, and generate the verification result of the verification operation based on the comparison result. Exemplary image comparison algorithms may include a pixel-by-pixel comparison algorithm, a histogram comparison algorithm, a structural similarity index (SSIM) algorithm, a mean squared error (MSE) algorithm (or a root mean squared error (RMSE) algorithm), a feature-based comparison algorithm, a deep learning-based comparison algorithm, a Hash-based comparison (perceptual hashing) algorithm, or the like, or any combination thereof. For instance, if the comparison result indicates a difference between the first sequence file and the second sequence file is less than a difference threshold, the processing device 140 may generate the successful verification result of the verification operation (e.g., a verification result indicating that the consistency validation is passed). Otherwise, the processing device 140 may generate the failing verification result of the verification operation (e.g., a verification result indicating that the consistency validation is failed).

In some embodiments, the processing device 140 may generate a final successful verification result of the verification operation when all of the compile validation, the parameter validation, and the consistency validation are passed.

By performing verification operation on the one or more MRI sequence elements, the conversion errors between the intermediate file and the one or more MRI sequence elements can be avoided, which can improve the accuracy of the conversion, further improving the accuracy of the MR scan.

If the failure verification result of the verification operation is obtained (i.e., the verification operation (e.g., at least one of the compile validation, the parameter validation, and the consistency validation) is not passed), the processing device 140 may modify the one or more MRI sequence elements. For example, the processing device 140 may re-perform operations 304 and 306 until the one or more MRI sequence elements pass the verification operation. As another example, the processing device 140 may remind the user to modify the first sequence file, such that the final successful verification result of the verification operation can be obtained. For instance, if the third value of the RF parameter exceeds the corresponding value range (e.g., a maximum value of the RF parameter of the MRI device), the processing device 140 may remind the user to decrease the third value of the RF parameter in the first sequence file.

If the successful verification result of the verification operation is obtained, the processing device 140 may proceed to operation 308.

In 308, the processing device 140 (e.g., the generation module 240) may generate hardware instructions based on the one or more MRI sequence elements.

The hardware instructions refer to control instructions to direct the hardware of the MRI device to perform specific operations.

In some embodiments, the hardware instructions may be generated based on the one or more MRI sequence elements in the second development environment. For example, the processing device 140 may compile the contents in the one or more MRI sequence elements in the second development environment, and generate the hardware instructions based on the contents in the one or more MRI sequence elements, such that the hardware of the MRI device can be directed to apply the one or more MRI sequence elements in an MR scan to be performed on the target subject.

In some embodiments, the processing device 140 may obtain updated information related to the MRI sequence in the first development environment, and update the hardware instructions directly in the second development environment based on the update information. For example, when the user updates the information (e.g., the gradient information, the RF information, the ADC information, the supplementary information, etc.) of the MRI sequence through the user interface of the terminal(s) 130 or the imaging device 110 in the first development environment, the processing device 140 may compile the updated information and update the hardware instructions in the second development environment based on the compiled information. As another example, when the verification result of the verification operation (e.g., the consistency validation) is failed, the user may input the updated information related to the MRI sequence in the first development environment or the second development environment. In some embodiments, the user may input the updated information by updating the first sequence file or the second sequence file.

By directly updating the hardware instructions in the second development environment based on the updated information, no additional operation needs to be performed (e.g., on the first file), which can simply the update process of the hardware instructions, thereby improving the efficiency of the hardware instruction generation.

In 310, the processing device 140 (e.g., the control module 250) may control the MRI device to execute the hardware instructions to apply the MRI sequence on the target subject.

For example, the processing device 140 may send the hardware instructions to the corresponding hardware of the MRI device, so as to direct the hardware to execute the hardware instructions, respectively. Correspondingly, the MR sequence may be applied to the subject and MR signals corresponding to the MRI sequence may be obtained. If the user updates the information of the MR sequence, an updated MR sequence may be applied to the target subject.

According to some embodiments of the present disclosure, by introducing the intermediate file, the one or more MRI sequence elements and the hardware instructions can be generated in the second development environment, even if the intermediate file is generated in the first development environment with distinct development language and development mode from the second development environment. This approach eliminates incompatibility between the first development environment and the second development environment, thereby reducing the complexity of MR application development and enhancing development efficiency. Furthermore, by generating the intermediate file in the first development environment, any development environment can be introduced to design the MRI sequence, which can provide more selections for the user, thereby improving the user experience. In addition, the user can design the intermediate file in the first development environment, which can reduce the user's dependence on the second development environment and learning and time cost associated with the second development environment, thereby further boosting the development efficiency.

FIG. 5 is a schematic diagram illustrating an exemplary process 500 for generating a first sequence file according to some embodiments of the present disclosure.

As illustrated in FIG. 5, a first sequence file 506 may include first value(s) 502 of one or more first parameters 508 related to an MRI sequence. The first value(s) 502 of at least one of the one or more first parameters 508 may be determined based on second values 504 of one or more second parameters 510 related to an MR scan to be performed based on the MRI sequence, and the first sequence file 506 may generated based on the first value(s) 502 of at least one of the one or more first parameters 508.

In some embodiments, the one or more first parameters 508 may include a gradient parameter, a radio frequency parameter, an ADC parameter, or the like, or any combination thereof, of the MRI sequence.

The one or more second parameters 510 may include a parameter related to an FOV or an ROI, a parameter related to a resolution, a parameter related to a scan time, or the like, or any combination thereof, of the MR scan. The FOV refers to a spatial dimension of the MR scan. The ROI refers to a scan region specified by a user. In some embodiments, the FOV may correspond to the ROI. The resolution refers to a resolution of an MRI image generated based on MR signals collected during the MR scan. The resolution may be used to describe the clarity of details in the MRI image. The scan time refers to a duration of the MR scan.

In some embodiments, the user may determine the second values 504 of the one or more second parameters 510 based on information of a target subject, and input the second values 504 through a user interface corresponding to a first development environment. Alternatively, the processing device 140 may determine the second values 504 of the one or more second parameters 510 based on the information of the target subject automatically. For example, the processing device 140 may input the information of the target subject into a parameter value determination model, and the parameter value determination model may output the second values 504 of the one or more second parameters 510. In some embodiments, the parameter value determination model may refer to a process or an algorithm used for generating the second values based on the information of the target subject. The parameter value determination model may be a trained machine learning model. In some embodiments, the parameter value determination model may be generated by training a first initial model using a plurality of first training samples. Each of the plurality of first training samples may include sample information of a sample subject and sample second values of the one or more second parameters corresponding to the sample subject. The sample information may be obtained from a medical database. The sample second values may be determined manually by the user and used as a training label.

In some embodiments, for each of the at least one first parameter 508, the first value 502 of the first parameter may be determined further based on a first corresponding relationship 512 between the first parameter and the one or more second parameters 510. The first corresponding relationship 512 may be represented by a first machine learning model, a table, a diagram, a mathematic function, etc. For example, the processing device 140 may input the second values 504 of the one or more second parameters 510 into the first machine learning model, and the first machine learning model may output the first value 502.

The first machine learning model may include a neural network (NN) model, a linear regression model, a long short term memory (LSTM) network model, a generative adversarial network (GAN) model, or the like, or any combination thereof. In some embodiments, the parameter value determination model may be generated by training a second initial model using a plurality of second training samples. Each of the plurality of second training samples may include sample first value of a sample first parameter and sample second values of the one or more second parameters corresponding to the sample first parameter. The plurality of second training samples may be obtained from a medical database.

In some embodiments, the one or more first parameters may include multiple first parameters, and a determination manner of first value(s) of part of the first parameters in the multiple first parameters may be different from a determination manner of first value(s) of remaining first parameter(s) in the multiple first parameters. Merely by way of example, the one or more first parameters 508 may include at least one first parameter and the remaining first parameter(s). First value(s) 514 of the remaining first parameter(s) may be input by the user in the first development environment. For each of the at least one first parameter, the first value 502 of the first parameter may be determined based on a second corresponding relationship 516 between the first parameter, the remaining first parameter(s), and the one or more second parameters 510. The second corresponding relationship 516 may be represented by a second machine learning model, a table, a diagram, a mathematic function, etc. For example, the processing device 140 may input the second values 504 of the one or more second parameters 510 and the first value(s) 514 into the second machine learning model, and the second machine learning model may output the first value 502. The second corresponding relationship 516 may be similar to the first corresponding relationship 512, which is not repeated herein.

In some embodiments, the processing device 140 may obtain updated information 518 of the one or more second parameters 510, and update the intermediate file 506 by updating the first values 502 of the one or more first parameters 508 included in the intermediate file 506 based on the updated information 518 of the one or more second parameters 510. The updated information refers to new value(s) of the second values 504 and/or modified information about the one or more second parameters 510. For example, the user may adjust a treatment plan of the target subject, and correspondingly adjust the MR scan. For instance, the user may input a new value of the FOV in the first development environment (e.g., via the user interface of the terminal(s) 130) or the second development environment (e.g., via the user interface of the imaging device 110), and the processing device 140 may update the intermediate file 506 by updating the first values 502 of the one or more first parameters 508 included in the intermediate file 506 based on the new value of the FOV.

According to some embodiments of the present disclosure, the first value(s) of at least one of the one or more first parameters can be generated based on the second value(s) of the one or more second parameters, so as to generate the first sequence file. Since the one or more second parameters are visual and readily comprehensible, the generation of the first sequence file can be simplified. In addition, by introducing the first corresponding relationship and/or the second corresponding relationship, the analysis of the big data may enable mining the complex relationship between the one or more first parameters and the one or more second parameters or the complex relationship between the MRI sequence and the MR scan, thus learning mechanism(s) of the first value determination. Therefore, the first value(s) of at least one of the one or more first parameters can be generated automatically or semiautomatically, which can reduce manual intervention, thereby improving the efficiency and accuracy of the generation of the first value(s) and the first sequence file.

Furthermore, the intermediate file can be directly updated based on the updated information of the one or more second parameters without updating the first sequence file, which can simply the updating process and improve the updating efficiency.

FIG. 6 is a schematic diagram illustrating an exemplary process 600 for generating an intermediate file according to some embodiments of the present disclosure.

As illustrated in FIG. 6, a type 620 of a first development environment 610 may be determined. A target script 640 corresponding to the type 620 of the first development environment 610 may be determined from a plurality of scripts 630 (e.g., a script 632, a script 634, a script 636, etc.) corresponding to different development environments. Further, the first sequence file 506 may be converted into an intermediate file 650 based on the target script 640.

The type 620 of the first development environment 610 may relate to a classification manner of the first development environment 610. For example, according to an establishment structure, the type 620 of the first development environment 610 may include a MATLAB development environment, a Visual Studio development environment, etc. As another example, according to an establishment language, the type 620 of the first development environment 610 may include a Java development environment, a Python development environment, a Visual Studio development environment, a C++ development environment, a Delphi development environment, etc.

In some embodiments, the processing device 140 may direct access the first development environment 610 to determine the type 620. Alternatively, the processing device 140 may determine the type 620 based on a suffix name of the first sequence file 506 generated in the first development environment 610. Since each suffix name corresponds to a unique type of a development environment, the type 620 can be determined based on the suffix name of the first sequence file 506.

In some embodiments, the plurality of scripts 630 may be developed in the second development environment in advance. For example, for each type of the first development environment 610, the processing device 140 may develop a script corresponding to the type of the first development environment 610.

A script may be configured for a corresponding development environment. For example, the script may be used to automatically convert the first sequence file generated in the corresponding development environment into the intermediate file. Merely by way of example, the processing device 140 may develop the script 632 configured for the Java development environment, the script 634 configured for the Python development environment, and the script 636 configured for the C++ development environment. If the type 620 of the first development environment 610 is determined as the Java development environment, the processing device 140 may determine the script 632 as the target script 640 from the scripts 632, 634, and 636, and then convert the first sequence file 506 into the intermediate file 650 based on the target script 640 (i.e., the script 632). For instance, information (e.g., the gradient information, the RF information, the ADC information, etc.) of an MRI sequence in the first sequence file 506 may be converted into the intermediate file 650 based on the script 632.

According to some embodiments of the present disclosure, by developing the plurality of scripts in the second development environment and determining the target script from the plurality of scripts, the first sequence file can be converted into the intermediate file automatically through the target script, which can reduce the labor workload and improve the efficiency of the generation of the intermediate file. In addition, the plurality of scripts are developed in the second development environment for different types of the first development environment, so that the first sequence file can be compatible with the second development environment based on the target script, which can avoid the incompatibility between the first development environment and the second development environment.

FIG. 7 is a schematic diagram illustrating an exemplary process 700 for generating one or more MRI sequence elements according to some embodiments of the present disclosure.

As illustrated in FIG. 7, a type 710 of an intermediate file 650 may be determined. A target interface 730 corresponding to the type 710 of the intermediate file 650 may be determined from a plurality of interfaces 720 (e.g., an interface 722, an interface 724, an interface 726, etc.) corresponding to different file types. Further, the intermediate file 650 may be converted file into one or more MRI sequence elements 750 using the target interface 730.

The type 710 of the intermediate file 650 may relate to a classification manner of the intermediate file 650. For example, according to a file format, the type 710 of the intermediate file 650 may include an image type, a table type, a text type, etc. As another example, according to an establishment language, the type 710 of the intermediate file 650 may include a Java file, a Python file, a Visual Studio file, a C++ file, a Delphi file, etc.

In some embodiments, the processing device 140 may determine the type 710 of the intermediate file 650 based on a suffix name of the intermediate file 650. It should be noted that the determination of the type based on the suffix name is provided for the purposes of illustration, and are not intended to limit the scope of the present disclosure.

In some embodiments, the plurality of interfaces 720 may be developed in the second development environment in advance. For example, for each type of the intermediate file, the processing device 140 may develop an interface corresponding to the type of the intermediate file.

An interface refers to a software component or an application program interface (API) developed in the second development environment to convert a corresponding file type of the intermediate file. For example, each of the plurality of interfaces 720 may be designed to recognize and process a specific type of the intermediate file.

Merely by way of example, the processing device 140 may develop the interface 722 configured for the image type, the interface 724 configured for the table type, and the interface 726 configured for the text type. If the type 710 of the intermediate file 650 is determined as the image type, the processing device 140 may determine the interface 722 as the target interface 730 from the interfaces 722, 724, and 726, and then convert the intermediate file 650 into the one or more MRI sequence elements 750 using the target interface 730 (i.e., the interface 722). For instance, information (e.g., the gradient information, the RF information, the ADC information, etc.) in the intermediate file 650 may be converted into a gradient element, an RF element, an ADC element, etc., using the interface 722, respectively.

According to some embodiments of the present disclosure, by developing the plurality of interfaces in the second development environment and determining the target interface from the plurality of interfaces, the intermediate file can be converted into the one or more MRI sequence elements automatically through the target interface, which can reduce the labor workload and improve the efficiency of the generation of the one or more MRI sequence elements. In addition, the plurality of interfaces are developed in the second development environment for different types of the intermediate file, so that the intermediate file can be compatible with the second development environment using the target interface, which can avoid the incompatibility between the first development environment and the second development environment.

FIG. 8 is a schematic diagram illustrating an exemplary process 800 for transmitting an intermediate file from a first development environment to a second development environment according to some embodiments of the present disclosure.

In 802, the processing device 140 (e.g., the obtaining module 260) may obtain value(s) of one or more parameters related to an MRI sequence.

The one or more parameters may include at least one first parameter. The at least one first parameter may include a gradient parameter, a radio frequency parameter, an ADC parameter, or the like, or any combination thereof.

In some embodiments, the one or more parameters may further include at least one second parameter. The at least one first parameter may include a parameter related to a field of view (FOV), a parameter related to a resolution, and a parameter related to a scan time, or the like, or any combination thereof, of an MR scan to be performed based on the MRI sequence.

Correspondingly, the value(s) may include at least one first value and/or at least one second value. More descriptions regarding the value(s) and the one or more parameters may be found elsewhere in the present disclosure. See, e.g., FIGS. 3 and 5 and relevant descriptions thereof.

In 804, the processing device 140 (e.g., the generation module 270) may generate an intermediate file based on the value(s) of the one or more parameters in a first development environment.

The intermediate file may be generated in the first development environment and be readable and convertible in a second development environment.

For example, the processing device 140 may directly generate the intermediate file based on the value(s) of the one or more parameters in the first development environment. As another example, the processing device 140 may generate a first sequence file based on the value(s) of the one or more parameters in the first development environment, and convert the first sequence file into the intermediate file in the first development environment. More descriptions regarding the generation of the intermediate file may be found elsewhere in the present disclosure. See, e.g., FIGS. 3 and 5-7, and relevant descriptions thereof.

In 806, the processing device 140 (e.g., the transmission module 280) may transmit the intermediate file from the first development environment to the second development environment.

For example, the processing device 140 may transmit the intermediate file from the first development environment to the second development environment through a cloud disk, a network disk, a local disk, a mobile disk, TCP/IP protocol, or the like, or any combination thereof. More descriptions regarding the transmission of the intermediate file may be found elsewhere in the present disclosure. See, e.g., FIG. 3 and relevant descriptions thereof.

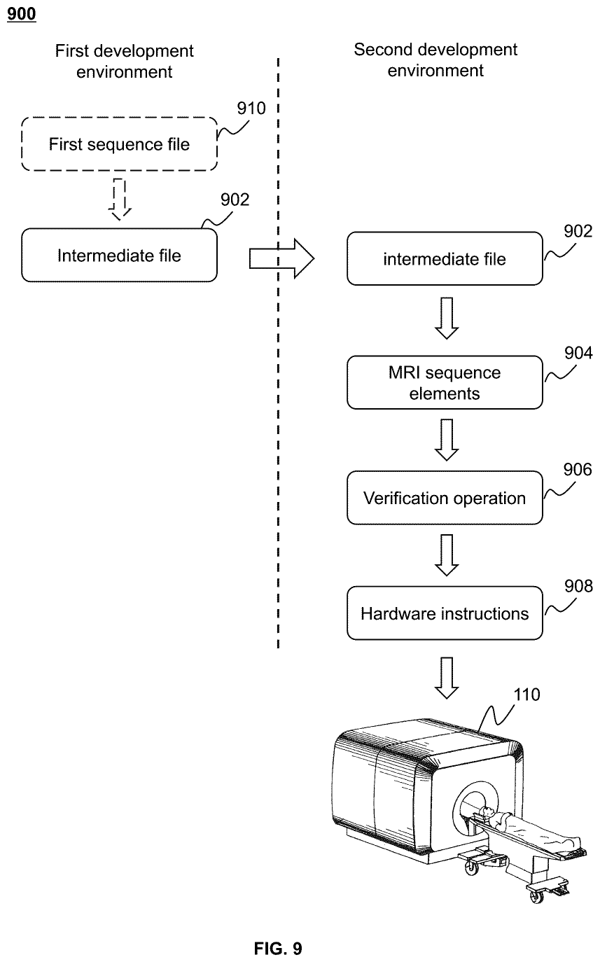

FIG. 9 is a schematic diagram illustrating an exemplary process 900 for MRI according to some embodiments of the present disclosure.

As illustrated in FIG. 9, an intermediate file 902 may be generated in a first development environment and be readable and convertible in a second development environment. For example, the intermediate file 902 may be converted from a first sequence file 910, and the first sequence file 910 may be generated in the first development environment.

In some embodiments, the intermediate file 902 may be transmitted to the second development environment. The intermediate file 902 may be converted into one or more MRI sequence elements 904 executable in the second development environment. A verification operation 906 may be performed on the one or more MRI sequence elements 904. In response to a successful verification result of the verification operation 906, hardware instructions 908 may be generated based on the one or more MRI sequence elements 904, and the MRI device 110 may be controlled to execute the hardware instructions 908 to apply an MRI sequence on a target subject.

It should be noted that the descriptions of the processes 300 and 500-900 are provided for the purposes of illustration, and are not intended to limit the scope of the present disclosure. For persons having ordinary skills in the art, various variations and modifications may be conducted under the teaching of the present disclosure. For example, the processes 300 and 500-900 may be accomplished with one or more additional operations not described, and/or without one or more of the operations discussed. Additionally, the order in which the operations of the processes 300 and 500-900 is not intended to be limiting. However, those variations and modifications may not depart from the protection of the present disclosure.

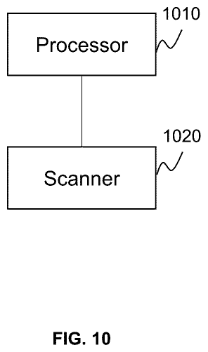

FIG. 10 is a schematic diagram illustrating an exemplary imaging device 1000 according to some embodiments of the present disclosure.

As illustrated in FIG. 10, an imaging device 1000 may include a processor 1010 and a scanner 1020. The processor 1010 may be configured to implement the functions and/or methods disclosed in the present disclosure. The scanner 1020 may be configured to generate or provide image data by scanning a target subject (or at least a portion of the target subject).

Merely for illustration, only one processor is described in FIG. 10. However, it should be noted that the computing device 1000 in the present disclosure may also include multiple processors. Thus operations and/or method steps that are performed by one processor as described in the present disclosure may also be jointly or separately performed by the multiple processors. For example, if the processor of the computing device 1000 in the present disclosure executes both operation A and operation B, it should be understood that operation A and operation B may also be performed by two or more different processors jointly or separately (e.g., a first processor executes operation A and a second processor executes operation B, or the first and second processors jointly execute operations A and B).

According to some embodiments of the present disclosure, a computing device is provided. The computing device may include a storage, a processor, and a computer application. When the computer application is executed by the processor, methods for MRI (e.g., the processes 300 and 500-900) disclosed in the present disclosure may be implemented.

According to some embodiments of the present disclosure, a computer-readable medium is provided. The computer-readable medium may store computer instructions. When the computer instructions are executed by a processor, methods for MRI (e.g., the processes 300 and 500-900) disclosed in the present disclosure may be implemented.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for magnetic resonance imaging (MRI), implemented on a computing device having at least one storage device storing a set of instructions, and at least one processor in communication with the at least one storage device, the method comprising:
obtaining an intermediate file related to an MRI sequence, the intermediate file being generated in a first development environment and being readable and convertible in a second development environment;
converting the intermediate file into one or more MRI sequence elements executable in the second development environment;
performing, in the second development environment, a verification operation on the one or more MRI sequence elements; and
in response to a successful verification result of the verification operation,
generating hardware instructions based on the one or more MRI sequence elements, and
controlling an MRI device to execute the hardware instructions to apply the MRI sequence on a target subject.

2. The method of claim 1, wherein the second development environment is a development environment of the MRI device, and the second development environment is different from the first development environment.

3. The method of claim 1, wherein the intermediate file includes gradient information, radio frequency information, and analog-to-digital converter (ADC) information of the MRI sequence.

4. The method of claim 1, wherein the intermediate file is converted from a first sequence file, the first sequence file being generated in the first development environment.

5. The method of claim 4, wherein
the first sequence file includes first values of one or more first parameters related to the MRI sequence, the one or more first parameters including at least one of: a gradient parameter, a radio frequency parameter, and an ADC parameter of the MRI sequence; and
the first value of at least one of the one or more first parameters is determined based on second values of one or more second parameters related to an MR scan to be performed based on the MRI sequence, the second values of the one or more second parameters being input by a user in the first development environment, and the one or more second parameters including at least one of: a parameter related to a field of view (FOV), a parameter related to a resolution, and a parameter related to a scan time of the MR scan.

6. The method of claim 5, further comprising:
obtaining updated information of the one or more second parameters input by the user in the first development environment; and
updating the intermediate file by updating the first values of the one or more first parameters included in the intermediate file based on the updated information of the one or more second parameters.

7. The method of claim 4, wherein the intermediate file is converted from the first sequence according a process including:
determining a type of the first development environment;
determining, from a plurality of scripts corresponding to different development environments, a target script corresponding to the type of the first development environment; and
converting the first sequence file into the intermediate file based on the target script.

8. The method of claim 1, wherein the converting the intermediate file into one or more MRI sequence elements executable by the second development environment includes:
determining a type of the intermediate file;
determining, from a plurality of interfaces corresponding to different file types, a target interface corresponding to the type of the intermediate file; and
converting the intermediate file into the one or more MRI sequence elements using the target interface.

9. The method of claim 1, wherein one or more MRI sequence elements executable by the second development environment include at least one of a data acquisition element, a gradient element, and a radio frequency element.

10. The method of claim 1, wherein the performing, in the second development environment, a verification operation on the one or more MRI sequence elements includes:
determining, in the second development environment, whether the one or more MRI sequence elements are implementable by the hardware of the MRI device.

11. The method of claim 1, wherein the performing, in the second development environment, a verification operation on the one or more MRI sequence elements includes:
generating, based on the one or more MRI sequence elements, a second sequence file;
displaying the second sequence file to a user via a user terminal;
obtaining feedback information with respect to the second sequence file input by the user via the user terminal; and
generating, based on the feedback information, a verification result of the verification operation.

12. The method of claim 1, further comprising:
obtaining updated information related to the MRI sequence in the first development environment; and
updating, based on the update information, the hardware instructions directly in the second development environment.

13. The method of claim 1, wherein the intermediate file includes template nodes for filling different types of information of the MRI sequence.

14. The method of claim 1, wherein the intermediate file is converted from a first sequence file, the first sequence file being generated in a third development environment different from the first development environment and the second development environment.

15. The method of claim 1, wherein parameters of one or more MRI sequence elements are determined based on parameters included in the intermediate file.

16. A system for magnetic resonance imaging (MRI), comprising:
at least one storage device including a set of instructions; and
at least one processor configured to communicate with the at least one storage device, wherein when executing the set of instructions, the at least one processor is configured to direct the system to perform operations including:
obtaining an intermediate file related to an MRI sequence, the intermediate file being generated in a first development environment and being readable and convertible in a second development environment;
converting the intermediate file into one or more MRI sequence elements executable in the second development environment;
performing, in the second development environment, a verification operation on the one or more MRI sequence elements; and
in response to a successful verification result of the verification operation,
generating hardware instructions based on the one or more MRI sequence elements, and
controlling an MRI device to execute the hardware instructions to apply the MRI sequence on a target subject.

17. The system of claim 16, wherein the intermediate file is converted from a first sequence file, the first sequence file being generated in the first development environment.

18. The system of claim 17, wherein
the first sequence file includes first values of one or more first parameters related to the MRI sequence, the one or more first parameters including at least one of: a gradient parameter, a radio frequency parameter, and an ADC parameter of the MRI sequence; and
the first value of at least one of the one or more first parameters is determined based on second values of one or more second parameters related to an MR scan to be performed based on the MRI sequence, the second values of the one or more second parameters being input by a user in the first development environment, and the one or more second parameters including at least one of: a parameter related to a field of view (FOV), a parameter related to a resolution, and a parameter related to a scan time of the MR scan.

19. The system of claim 18, the operations further comprising:
obtaining updated information of the one or more second parameters input by the user in the first development environment; and
updating the intermediate file by updating the first values of the one or more first parameters included in the intermediate file based on the updated information of the one or more second parameters.

20. A non-transitory computer readable medium, comprising executable instructions that, when executed by at least one processor, direct the at least one processor to perform a method, the method comprising:
obtaining an intermediate file related to an MRI sequence, the intermediate file being generated in a first development environment and being readable and convertible in a second development environment;
converting the intermediate file into one or more MRI sequence elements executable in the second development environment;
performing, in the second development environment, a verification operation on the one or more MRI sequence elements; and
in response to a successful verification result of the verification operation,
generating hardware instructions based on the one or more MRI sequence elements, and
controlling an MRI device to execute the hardware instructions to apply the MRI sequence on a target subject.
